(19) United States
(12) Patent Application Publication
(10) Pub. No.: US 2025/0261907 A1
(43) Pub. Date: Aug. 21, 2025

(54) BIOLOGICAL INFORMATION DETECTOR, BIOLOGICAL INFORMATION PROCESSING DEVICE, AND BIOLOGICAL INFORMATION PROCESSING METHOD

(71) Applicant: Yoshihiro SEKINE, Aichi (JP)

(72) Inventor: Yoshihiro SEKINE, Aichi (JP)

(21) Appl. No.: 19/051,198

(22) Filed: Feb. 12, 2025

(30) Foreign Application Priority Data
Feb. 15, 2024 (JP) 2024-021213

(51) Int. Cl. A61B 5/00

(52) U.S. Cl. CPC A61B 5/721; A61B 2562/0219; A61B 2562/063

(57) ABSTRACT

A biological information detector includes multiple sensors to measure body motion of a body and output body motion information, a sensor between the multiple sensors to measure biological information of the body and output the biological information, and a detector. The detector detects the biological information based on the biological information output from the sensor and the body motion information output from the multiple sensors.

### CROSS-REFERENCE TO RELATED APPLICATIONS

This patent application is based on and claims priority pursuant to 35 U.S.C. § 119(a) to Japanese Patent Application No. 2024-021213, filed on Feb. 15, 2024, in the Japan Patent Office, the entire disclosure of which is hereby incorporated by reference herein.

### BACKGROUND

### Technical Field

Embodiments of the present disclosure relate to a biological information detector, a biological information processing device, and a biological information processing method.

### Related Art

When the biological information of a human body in daily life is measured as an observation signal by a sensor such as a wearable device, the observation signal may include body motion noise generated in accordance with the movement of the body. It is known that body motion noise is included in an observation signal detected by a pulse wave sensor. For example, when such a pulse wave sensor is a biological information detector attached to the wrist of a person to be measured, a motion such as an arm swinging motion is a factor to generate body motion noise. Such an arm swinging motion can be detected by a body motion sensor such as an acceleration sensor. For this reason, if the biological information detector is operated as a single sensor and noise reduction processing is performed using the body motion data detected by the body motion sensor, the body motion noise caused by the arm swinging motion can be reduced to a certain extent if, for example, the arm swinging motion is not vigorous or the arm is not swung periodically. The above-described noise reduction processing is, for example, adaptive filter processing.

### SUMMARY

In an embodiment of the present disclosure, a biological information detector includes multiple sensors to measure body motion of a body and output body motion information, a sensor between the multiple sensors to measure biological information of the body and output the biological information, and a detector. The detector detects the biological information based on the biological information output from the sensor and the body motion information output from the multiple sensors.

In another embodiment of the present disclosure, a biological information processing device includes circuitry. The circuitry receives body motion information output from multiple sensors to measure body motion of a body at multiple positions, receives biological information of the body output from the sensor to measure the biological information at a position between the multiple sensors, estimates the body motion information at the position of the sensor based on the body motion information measured by the multiple sensors, and detects the biological information based on the biological information measured by the sensor at the position and the body motion information estimated at the position.

In still another embodiment of the present disclosure, there is provided a method for processing biological information. The method includes measuring body motion of a body, measuring biological information of the body, outputting the biological information, and estimating the body motion information. The measuring body motion measures the body motion of a body at multiple positions and outputting body motion information. The measuring biological information measures biological information of the body at a position between the multiple positions and outputting the biological information. The estimating the body motion information estimates the body motion information at the position, at which the biological information is measured, based on the body motion information measured at the multiple positions. The detecting the biological information detects the biological information based on the biological information measured at the position and the body motion information estimated at the position.

### BRIEF DESCRIPTION OF THE DRAWINGS

A more complete appreciation of the disclosure and many of the attendant advantages and features thereof can be readily obtained and understood from the following detailed description with reference to the accompanying drawings, wherein:

FIG. 1A is a block diagram illustrating a configuration of a body motion noise reduction system including a biological information detector according to an embodiment of the present disclosure;

FIGS. 1B and 1C are diagrams each illustrating an arrangement of sensors of the body motion noise reduction system of FIG. 1A, according to an embodiment of the present disclosure;

FIGS. 2A, 2B, and 2C are diagrams each illustrating a process of detecting biological information performed by the body motion noise reduction system of FIG. 1A, according to an embodiment of the present disclosure;

FIGS. 3A, 3B, and 3C are diagrams each illustrating a process of detecting biological information performed by the body motion noise reduction system of FIG. 1A, according to an embodiment of the present disclosure;

FIG. 4 is a circuit diagram illustrating a detection process to detect the biological information performed by the body motion noise reduction system of FIG. 1A, according to an embodiment of the present disclosure;

FIGS. 5A, 5B, 5C, and 5D are diagrams each illustrating a detection process to detect the biological information performed by the body motion noise reduction system of FIG. 1A, according to an embodiment of the present disclosure;

FIGS. 6A, 6B, 6C, and 6D are diagrams each illustrating a detection process to detect the biological information performed by the body motion noise reduction system of FIG. 1A, according to an embodiment of the present disclosure;

FIGS. 7A, 7B, and 7C are diagrams each illustrating a process of detecting the biological information performed by the body motion noise reduction system of FIG. 1A, according to an embodiment of the present disclosure; and

FIGS. 8A, 8B, and 8C are diagrams each illustrating a detection process to detect the biological information performed by a body motion noise reduction system according to a modification of embodiments of the present disclosure.

The accompanying drawings are intended to depict embodiments of the present disclosure and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result.

Referring now to the drawings, embodiments of the present disclosure are described below. As used herein, the singular forms “a,” “an,” and “the” are intended to include the plural forms as well, unless the context clearly indicates otherwise.

Embodiments of a biological information detector, a biological information processing device, and a biological information processing method are described in detail with reference to the accompanying drawings.

First, a description is given of an example of a biological information detector, a biological information processing device, and a biological information processing method according to an embodiment of the present disclosure. A wearable device that senses sensing data such as vital data is affected by a body motion associated with the movement of a human body when detecting the vital data. Accordingly, unnecessary noise, i.e., body motion noise, is mixed in the detected vital data due to the influence of the body motion. As a result, the measurement accuracy of the sensing data may be reduced and stable measurement may not be performed. In addition, even if an optimal position of the wearable device can be found for detecting the sensing data to reduce the influence of the body motion noise, conversely, the detection sensitivity of the wearable device to detect the sensing data itself is also reduced. Thus, finding the optimal position for the wearable device and the detection sensitivity of the wearable device have a trade-off relation. Thus, it is difficult to detect the sensing data stably. Further, even if filtering processing using signal processing is performed to reduce the frequency of the body motion noise, the body motion noise cannot be removed when the frequency bands of the sensing data and the body motion noise are close to each other.

For this reason, the biological information detector of the present embodiment includes a biological information sensor to detect and measure the biological information and multiple body motion information sensors to detect and measure the body motion noise. The multiple body motion information sensors are arranged at a predetermined interval such that the body motion information sensors sandwich the biological information sensor and arranged side by side around an object to be sensed. The biological information detector can calculate backward and estimate the body motion noise that is detected by the biological information sensor based on the body motion noise detected by the body motion information sensors. Such a configuration allows the biological information detector to calculate a difference between the body motion noise and the biological information. In so doing, the biological information detector reduces the influence of the body motion noise and detects and measures the biological information with high accuracy. The body motion noise detected by the biological information sensor and the body motion noise detected by the body motion information sensors have a different noise level. For this reason, the biological information detector employs the biological information sensor and the body motion information sensors as hardware components to directly remove the difference of the noise levels based on the difference between the body motion noise detected by and superimposed onto the biological information sensor and the body motion noise detected by and superimposed onto the body motion information sensors.

In other words, in the biological information detector of the present embodiment, the biological information sensor that detects the biological information such as a pulse wave from a living organism is attached to a human body. The body motion information sensors that have characteristics similar to the biological information sensor are arranged so as to sandwich a position (preferably, a position at which detection of the biological information is hardly interfered) at which the biological information sensor detects the biological information at predetermined intervals. The biological information detector causes the body motion data sensors to detect a body motion noise having a pulse wave pattern similar to, and at the same time, having a different magnitude from the body motion noise superimposed onto the biological information detected by the biological information sensor. The biological information detector uses the body motion noise having the different magnitude to remove the body motion noise superimposed onto the biological information detected by the biological information sensor. In the present embodiment, even when the body motion information sensors are close to the biological information sensor that detects the biological information and the body motion noise detected by the body motion information sensors are equal to the body motion noise detected by the biological information sensor, the biological information detector can remove the body motion noise that is superimposed onto the biological information detected by the biological information sensor.

In the present embodiment, the body motion information sensors are arranged at a predetermined interval (known interval) with respect to the biological information sensor, and the noise level of the body motion noise detected by the biological information sensor is predicted by the relation, i.e., ratio of distance between the biological information sensor and the body motion information sensors, which is calculated from the predetermined interval. Thus, the difference between the body motion noise detected by the biological information sensor and the body motion noise detected by the body motion information sensors is obtained. By so doing, the biological information detector can efficiently remove the body motion noise with a simple hardware configuration. In the present embodiment, even if the body motion information sensors are close to the biological information sensor and the body motion noise detected by the body motion information sensors are similar to the body motion noise detected by the biological information sensor, the body motion noises detected by the body motion information sensors are added, then divided to half since the body motion information sensors are disposed at equal intervals from the biological information sensor. For this reason, the body motion noise detected by the biological information detector is estimated to be equivalent to the body motion noise detected by the body motion noise detected by the body motion information sensors.

Such a configuration as described above allows the biological information detector to detect the biological information in a stable and highly accurate manner without being affected by the body motion noise, even when the body motion noise having a frequency (periodicity) close to the biological information is superimposed onto the biological information. Further, the biological information detector can sufficiently reduce the body motion noise only by the arrangement of the biological information sensor and the body motion information sensors and a hardware circuit or by a simple configuration without employing any device, even if signal processing is hardly performed. Accordingly, the biological information detector can detect the biological information with high accuracy. Further, complicated signal processing is not necessary. Accordingly, the biological information detector can perform real-time processing.

In addition, the biological information detector can estimate the body motion noise superimposed onto the biological information detected by the biological information sensor almost regardless of directions in which the body motion noises are generated. Accordingly, the biological information detector can detect the biological information with high accuracy. Application examples of the biological information detector includes a case as follows. For example, each of the body motion information sensors that are arranged around the biological information sensor includes multiple arrays of sensors. By so doing, the biological information detector can estimate the body motion noises generated from various directions. Accordingly, the biological information detector can reduce the influence of distortion generated from various directions and the angle of the distortion from which the distortion are detected. As a result, the biological information detector can remove the body motion noise with high accuracy and detect the biological information with high accuracy.

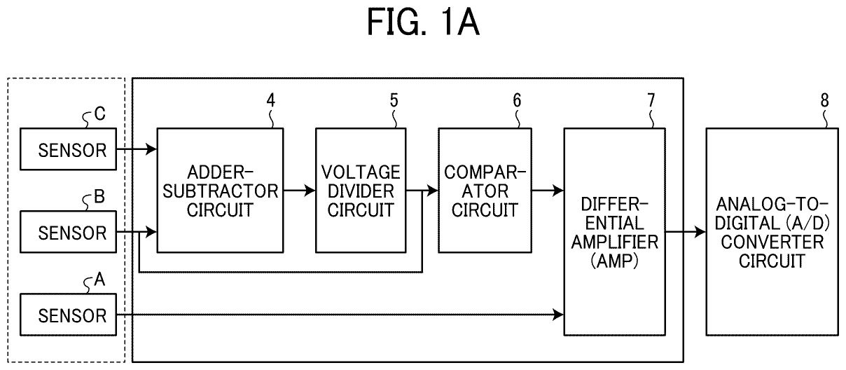

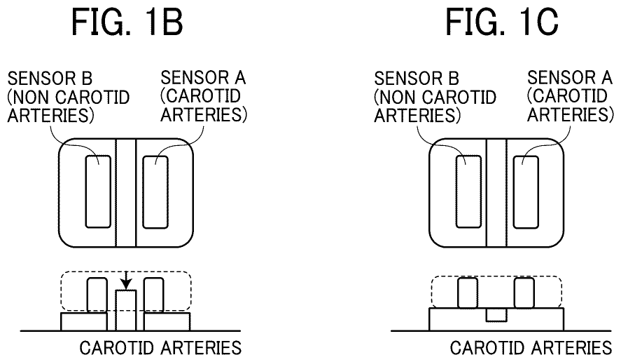

Next, specific examples of the biological information detector, the biological information processing device, and the biological information processing method according to embodiments of the present disclosure are described with reference to FIGS. 1A, 1B, 1C, 2A, 2B, 2C, 3A, 3B, 3C, 4, 5A, 5B, 5C, 5D, 6A, 6B, 6C, 6D, 7A, 7B, 7C, 8A, 8B, and 8C. FIG. 1A is a block diagram illustrating a configuration of a body motion noise reduction system including the biological information detector according to an embodiment of the present disclosure.

FIG. 1B is a diagram illustrating an arrangement of sensors of the body motion noise reduction system, according to an embodiment of the present disclosure. As illustrated in FIG. 1A, the body motion noise reduction system according to the present embodiment includes sensors A, B, and C, an adder-subtractor circuit 4, a voltage divider circuit 5, a comparator circuit 6, a differential amplifier 7, and an analog-to-digital (A/D) converter circuit 8.

The sensor A is an example of a biological information sensor that detects and measures the biological information. The sensors B and C are examples of multiple body motion information sensors that detect and measure the body motion noise (body motion information). The sensors B and C are arranged such that the sensors B and C sandwich the sensor A. Such a configuration as described above allows the biological information detector to accurately estimate the body motion noise at the position of the sensor A based on the outputs of the sensors B and C.

In the present embodiment, the sensors A, B, and C are, for example, body motion sensors, heart rate sensors, and pulse wave sensors.

The body motion sensor is, for example, an acceleration sensor, or a pressure sensor, and is attached to the forehead, the ear, the neck of a person to be measured, to monitor the motion of the person to be measured. Examples of motions of the body to be monitored include swallowing saliva, sneezing, yawning, deep breathing, and prying. The body motion sensor may be a wearable sensor of a hair band type, a neckband type, or a patch type.

The heart rate sensor and the pulse wave sensor may be of any type that measures an electrocardiogram or a pulse wave. The body motion sensor may be, for example, an electrocardiogramonitor that causes two or greater electrodes to contact a body surface to obtain an electrocardiogram from a potential difference, or a photoplethysmographic measurement sensor that irradiates a skin surface with light to measure a reflected wave or transmitted light. Alternatively, the body motion sensor may be a wearable sensor in which an acceleration sensor or a pressure sensor is attached to the skin immediately above the artery to measure the pulse wave.

Examples of wearable pulse wave sensors include a clip type sensor attached to a fingertip, a wrist type sensor wound around a wrist, an earring type sensor attached to an earlobe, and a patch type sensor attached to a temple.

A description is given of an example of the arrangement of the sensors A and B below with reference to FIG. 1B. An example of the arrangement of the sensors A and B is described below. However, the sensors A and C may be arranged in a similar manner.

For example, in the body motion noise reduction system, an absorber or a convex-shaped medium that hardly conveys distortion due to, for example, a pulse wave, may be disposed between the sensor A and the sensor B. Such a configuration can reduce the body motion noise due to the structure of the sensor A and the sensor B. Alternatively, for example, as illustrated in FIG. 1C, the body motion noise reduction system may have a groove between the sensor A and the sensor B, and an absorber that hardly conveys distortion may be disposed in the groove.

The sensors A, B, and C may be elements divided from a same element or integrated as a single component. Desirably, the sensors A, B, and C have, for example, a similar sensitivity and temperature characteristics. Such a configuration allows the sensors A, B, and C to cope with the temperature change, variation among the sensors A, B, and C and aging of the sensors A, B, and C. Accordingly, the body motion noise reduction system can stably detect the biological information with high accuracy. In addition, the body motion noise reduction system can remarkably reduce the influence caused by the difference of detection sensitivity among the sensors A, B, and C with respect to the distortion caused by the body motion.

The adder-subtractor circuit 4 adds outputs of the sensor B and the sensor C. Alternatively, the adder-subtractor circuit 4 calculates the difference between the output of the sensor B and the output of the sensor C.

The voltage divider circuit 5 divides a value obtained by adding the outputs of the sensor B and the sensor C based on the distance or the ratio of the distance from the sensor A to each of the sensors B and C. For example, the voltage divider circuit 5 is a variable resistor that adjusts the output (signal level) of the adder-subtractor circuit 4.

The comparator circuit 6 is an example of an estimation unit that estimates the body motion noise at the position of the sensor A based on the outputs of the sensors B and C. In the present embodiment, the comparator circuit 6 estimates the body motion noise at the position of the sensor A based on the distances from the sensor A to the sensors B and C or the ratio of the distances from the sensor A to the sensors B and C. Accordingly, the body motion noise reduction system can accurately estimate the body motion noise at the position of the sensor A based on the distances or the ratio of the distances from the sensor A to the two sensors B and C.

The differential amplifier 7 is an example of a detector that detects the biological information based on the output of the sensor A and the body motion noise estimated by the comparator circuit 6. The differential amplifier 7 estimates the body motion noise at the position of the sensor A based on the outputs of the sensors B and C. By so doing, the differential amplifier 7 can accurately detect the biological information. The differential amplifier 7 is an example of a correction unit that detects the difference between the output of the sensor A and the outputs of the sensors B and C at predetermined intervals and corrects the output of the sensor A.

Accordingly, the body motion noise reduction system can allow the sensors B and C to follow the aging of the sensor A. Specifically, variable elements such as resistors are employed for the circuit of, for example, the differential amplifier 7, and the variable elements can be calibrated. By so doing, the differential amplifier 7 can reduce the influence of the aging and the positional deviation of the sensors A, B, and C with respect to the initial values. Accordingly, the body motion noise reduction system can stably detect biological information with high accuracy.

The A/D converter circuit 8 performs A/D conversion processing with respect to the waveform of the biological information detected by the differential amplifier 7.

In other words, the adder-subtractor circuit 4, the voltage divider circuit 5, the comparator circuit 6, and the differential amplifier 7 are each an example of a detector that detects the biological information based on the outputs (biological information) of the sensor A and the outputs (body motion noise) of the sensors B and C. Accordingly, the body motion noise reduction system can accurately detect the biological information based on the output of the sensor A and the outputs of the sensors B and C. In the present embodiment, the detector is a circuit unit including the adder-subtractor circuit 4, the voltage divider circuit 5, the comparator circuit 6, and the differential amplifier 7. However, the detector may be a computer.

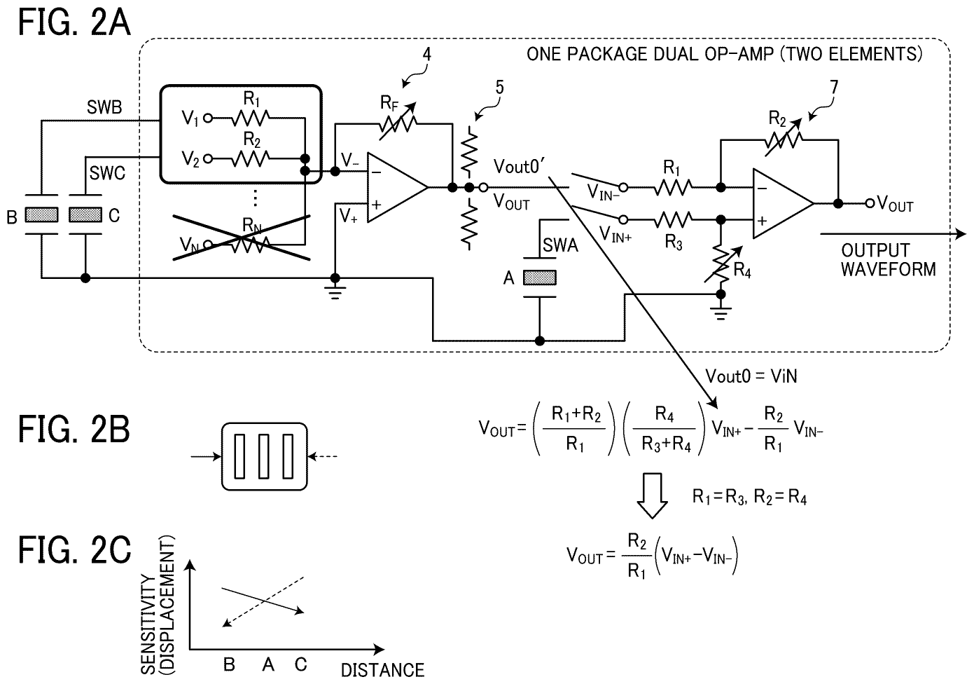

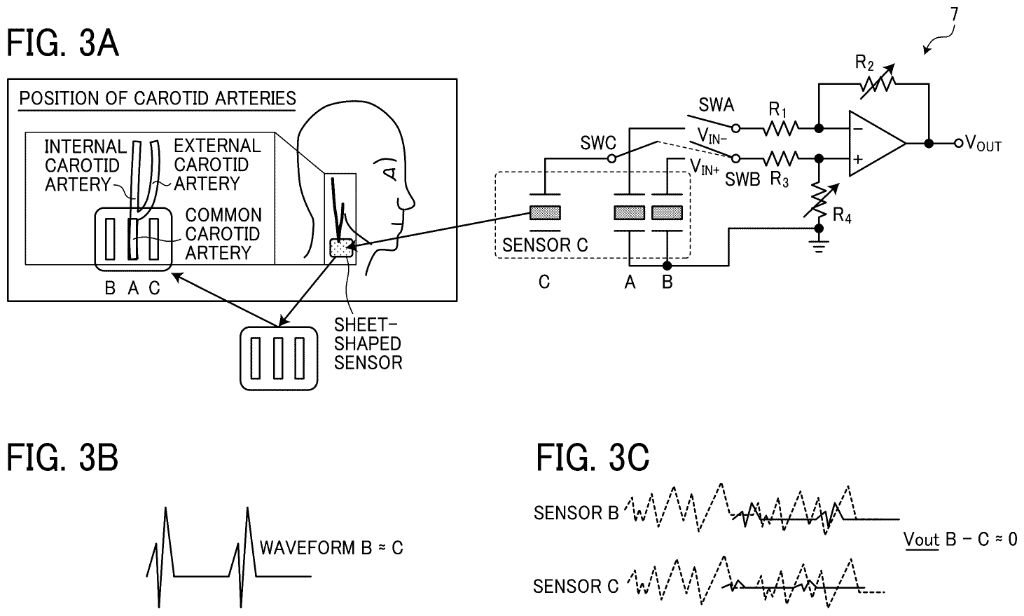

FIGS. 2A, 2B, 2C, 3A, 3B, and 3C are diagrams each illustrating a process of detecting the biological information performed by the body motion noise reduction system, according to an embodiment of the present disclosure. In the present embodiment, as illustrated in FIG. 3A, the body motion noise reduction system includes the sensors A, B and C. The sensor A is an example of a sensor that detects the biological information at the position of the carotid artery. The sensors B and C are the multiple sensors (for example, two) arranged adjacent to the sensor A such that the sensors B and C sandwich the sensor A with respect to an object to be sensed (for example, a carotid artery), which is an object to be sensed for detecting the biological information.

The sensors B and C are arranged adjacent to the sensor A such that the sensors B and C sandwich the sensor A. For this reason, distortion occurs around the object to be sensed. The body motion noise attenuates in the vicinity of the surface of the object to be sensed, i.e., in the distance between the sensor B and the sensor C, in a direction in which the body motion noise is generated, when the surface of the object to be sensed is deformed due to the stress of the distortion. The body motion noise reduction system uses the amount of attenuation of the body motion noise, to estimate the body motion noise superimposed onto the biological information of the object to be sensed, for example, carotid arteries, and obtains the difference, such as the biological information detected by differential amplifier 7, between the estimated body motion noise and the biological information of the object to be sensed. Such a configuration allows the body motion noise reduction system to reduce the influence of the body motion noise only by the sensors as the hardware components.

In addition, the body motion noise reduction system preliminarily calculates back the noise level of the sensor A that detects the biological information based on the transmission loss of the body motion noise due to the stress of the sensors B and C sandwiching the sensor A. Accordingly, the body motion noise reduction system can subtract the body motion noise only by the adder-subtractor circuit 4 and the parameter constant of the adder-subtractor circuit 4. In the example illustrated in FIGS. 2A, 2B, and 2C, the interval between the sensors A and B and the interval between the sensors A and C are equal. The body motion noise reduction system estimates that half of the sum of the outputs of the sensors B and C is the body motion noise superimposed onto the sensor A, and sets the estimated body motion noise as the parameter of the circuit constant. The differential amplifier 7 subtracts the body motion noise from the biological information and outputs only the biological information as the sensing data.

In other words, in the body motion noise reduction system, after the adder-subtractor circuit 4 as an initial stage of the circuit, adds the outputs of the sensors B and C, the voltage divider circuit 5 divides the added outputs by the coefficient of one half (½), and the differential amplifier 7 obtains the difference between the divided output and the output of the sensors A. By so doing, the body motion noise reduction system subtracts and removes the body motion noise by the hardware configuration.

When distortion occurs from around the sensor B, displacement (sensitivity) due to the distortion is attenuated in the order of the sensor B, the sensor A, and the sensor C in accordance with the distance, as illustrated in FIG. 2C. Accordingly, the displacement that occurs in the output of the sensor A is calculated back from the displacement of the outputs of the sensors B and C. When the above description is illustrated by a circuit in FIG. 2A, the displacement of the output (SWB in FIG. 2A) of the sensor B is added to the differential amplifier 7, and the displacements of the outputs attenuated by the sensors B to C are added, and then divided. The divided outputs are induced as a difference Vout 0′ to the differential amplifier 7 together with the output of the sensors A. By so doing, only the body motion noise is subtracted only by the configuration of the sensors A, B, an C and the configuration of the circuit as the hardware components. Vout is waveform data that includes only the biological information.

As described above, the body motion noise reduction system includes the sensors A, B, and C arranged at predetermined intervals (see FIG. 2B) and the simple hardware circuit to detect multiple pieces of the biological information. Accordingly, the body motion noise reduction system can reduce the influence of the body motion noise without performing complicated digital processing or signal processing on the biological information.

In addition, the body motion noise, which is non-biological information and unnecessary when detecting the biological information, has a frequency (periodicity) close to a signal of the biological information. For this reason, the body motion noise cannot be sufficiently removed only by a simple filter processing, and the signal processing in a complicated time region is needed. In this case, real-time processing cannot be performed in real time. Thus, detection performance of such a body motion noise reduction system to detect the biological information is deteriorated. By contrast, the body motion noise reduction system of the present embodiment can perform a highly accurate and stable real-time processing by the simple hardware of only the adder-subtractor circuit 4 and the multiple sensors A, B, and C that detect the biological information.

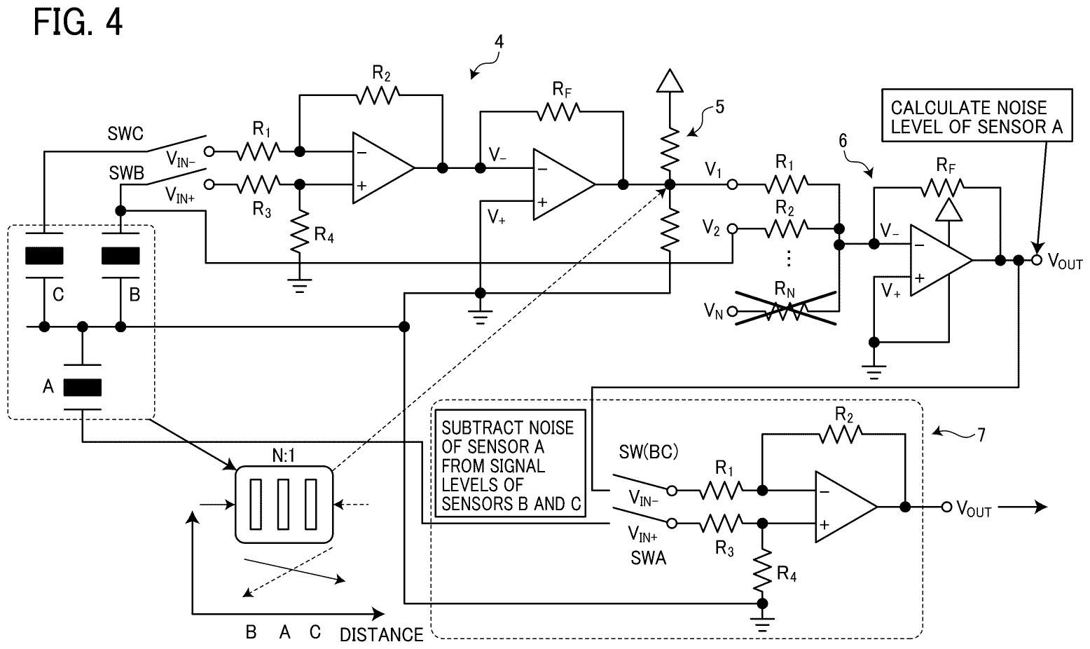

FIG. 4 is a circuit diagram illustrating a detection process to detect the biological information performed by the body motion noise reduction system, according to an embodiment of the present disclosure. In the example illustrated in FIGS. 2A, 2B, and 2C, regardless of directions in which the stress is generated, one half of the value obtained by adding the outputs of the sensor B and the sensor C is output as the noise level of the sensor A. Thus, the body motion noise is removed by the difference from the output of the sensor A. The example illustrated in FIG. 4 is a circuit configuration of the body motion noise reduction system. In this example, the sensors B and C that arranged adjacent to the sensor A such that the sensors B and C sandwich the sensor A, are not disposed symmetrically with respect to the sensor A for sensing the biological information. In other words, the sensors B and C are arranged so as to face each other asymmetrically across the sensor A.

The body motion noise reduction system obtains a difference between the output of the sensor B and the output of the sensor C. Subsequently, the body motion noise reduction system divides the difference by a coefficient ratio, i.e., a coefficient value calculated from a ratio associated with the interval between the sensors B and C, and offsets the output of the sensor B by the divided difference (waveform). Thus, the body motion noise reduction system finally outputs the difference between the output of the sensor B and the output of the sensor A. The real-time performance of the body motion noise reduction system is slightly reduced. However, when the sensor B is adversely affected by the biological information of the sensor A rather than the biological information of the sensor C due to, for example, the influence of the motion of the body, particularly, due to the individual variability among persons to be measured, the sensor B can be moved away from the sensor A by a necessary distance.

Specifically, the body motion noise reduction system divides the output, which at a ratio (N:1) based on the distance between the sensor B and the sensor A and the distance between the sensor C and the sensor A after the and outputs the divided output, and obtains the difference between the output waveform (Vout) obtained by adding the waveform of the sensor B as an offset to the output and the output of the sensor A. By so doing, reducing and removing the body motion noise included in the output of the sensor A.

The flow of electricity is as follows. First, a switch B (SWB) and a switch C (SWC) are turned on. Immediately thereafter, the SWC is turned off and a switch A (SWA), the SWB, and the SWC are turned on.

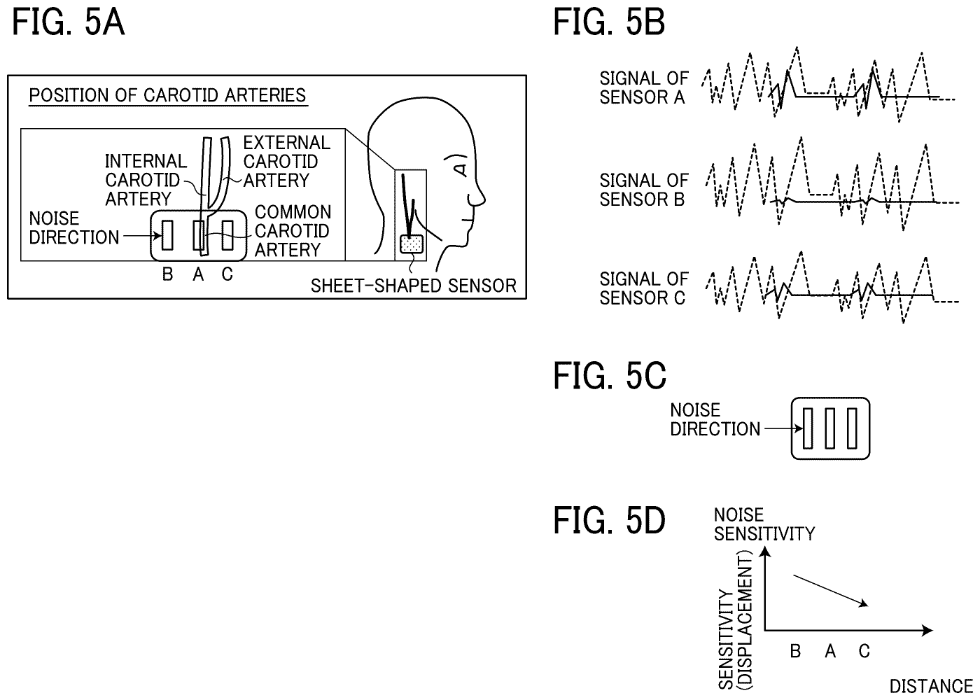

FIGS. 5A, 5B, 5C, and 5D are diagrams each illustrating a detection process to detect the biological information performed by the body motion noise reduction system, according to an embodiment of the present disclosure. As illustrated in FIG. 5A, when the center of the sensor A that detects the biological information is shifted from the object to be sensed (in this case, common carotid artery), the positions of the sensors B and C that sandwich the sensor A are also relatively shifted. The body motion noise reduction system of the present embodiment can reduce the body motion noise itself. Accordingly, the decrease in signal-to-noise (S/N) ratio relative to the positional deviation of the sensors A, B, and C is small. In the case of FIG. 5A, the relative sensitivity of the body motion noise reduction system changes in accordance with directions in which the positions of the sensors A, B, and C are shifted. However, the body motion noise reduction system calculates back the body motion noise superimposed onto the sensor A as the body motion noise determined by the distance between the sensors B and C with respect to the sensor A. Thus, the body motion noise reduction system can finally remove the body motion noise by the difference from the sensor A. However, in the body motion noise reduction system according to the present embodiment, the S/N ratio is slightly reduced.

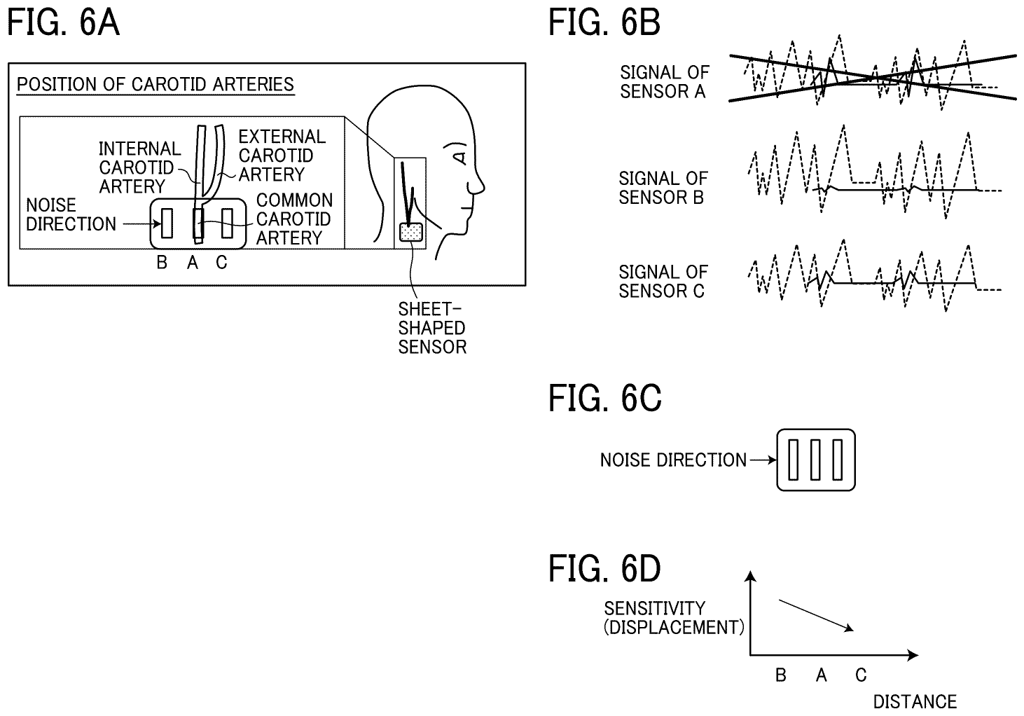

FIGS. 6A, 6B, 6C, and 6D are diagrams each illustrating a detection process to detect the biological information performed by the body motion noise reduction system, according to the present embodiment. As illustrated in FIG. 6A, the sensor A that detects the biological information is not included in the body motion noise reduction system of the present embodiment, and the body motion noise reduction system can detect the biological information even with only the sensors B and C. However, in this case, digital processing is performed, and complicated signal processing is inevitable. Moreover, real-time processing is difficult. Thus, stable measurement of the biological information other than the S/N ratio is difficult.

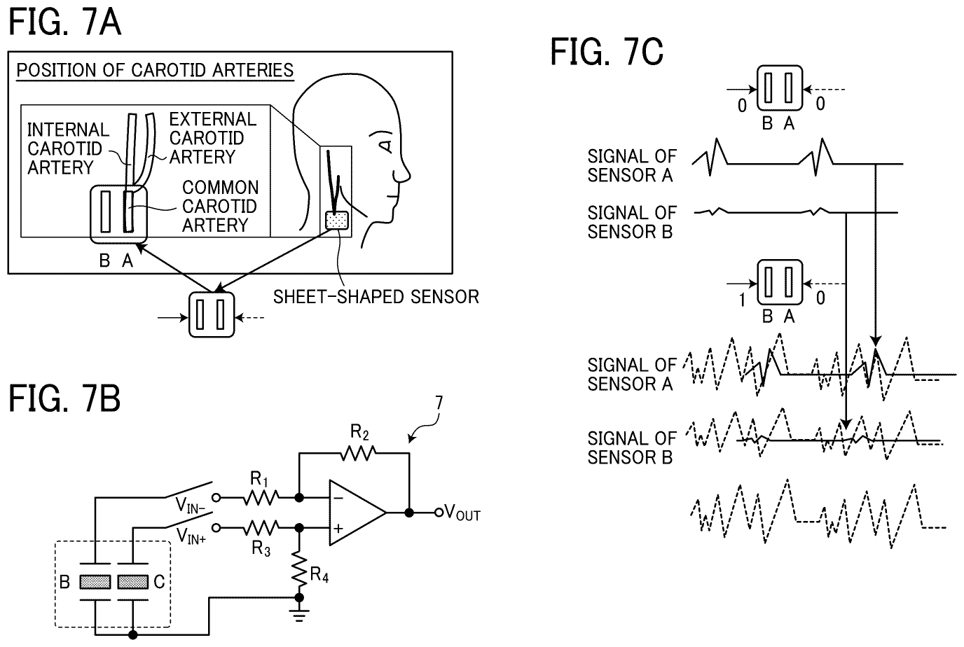

FIGS. 7A, 7B, and 7C are diagrams each illustrating a process of detecting the biological information performed by the body motion noise reduction system, according to an embodiment of the present disclosure. As illustrated in FIG. 7A, when only the sensor B is present around the sensor A that detects the biological information, the body motion noise reduction system can detect the biological information even only by a difference between the outputs of the sensors A and B in a detection process similar to the above-described detection process. However, with only the difference between the outputs of the sensors A and B, when the noise levels of the sensors A and B are equal, the S/N ratio is favorable. However, the levels of the body motion noise detected by the sensors A and B due to distortion generated are different depending on a location, i.e., a distance from the body motion noise. Accordingly, in most cases, the noise levels are not equal, and the S/N ratio is reduced even if the biological information can be detected. In addition, due to the positional deviation of the sensors A and B, the sensing level of the biological information is further reduced, and the body motion noises detected by the sensors A and B are also asymmetric. As a result, the S/N ratio is further reduced.

As described above, the body motion noise reduction system of the present embodiment estimates the body motion noise at the position of the sensor A based on the outputs of the sensors B and C. By so doing, the body motion noise reduction system can reduce the influence of the body motion noise included in the sensing data of the biological information. Thus, the body motion noise reduction system can detect the biological information with high accuracy.

### Modification

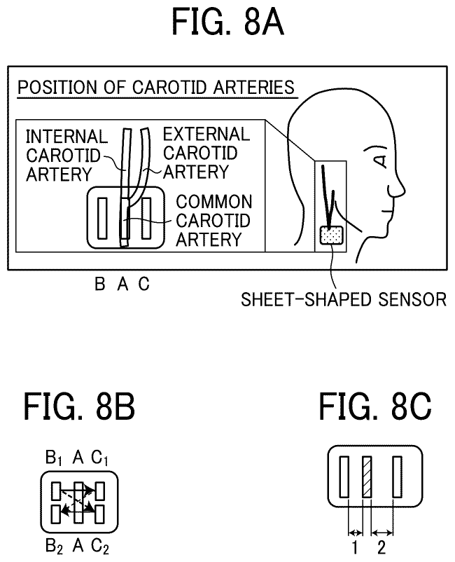

FIGS. 8A, 8B, and 8C are diagrams each illustrating a detection process to detect the biological information performed by the body motion noise reduction system according to a modification of the above embodiments of the present disclosure. As illustrated in FIG. 8B, the body motion noise reduction system of the modification includes multiple sensors B1, B2, C1, and C2 arranged around the sensor A for detecting the biological information such that the sensors B1, B2, C1, and C2 surround the sensor A. The sensors B1, B2, C1, and C2 are formed of a similar element and have subdivided structure. Accordingly, the body motion noise reduction system of the modification can detect the biological information favorably even when the surface of the body moves in various directions due to the movement of the body.

The sensitivity of the sensors B and C arranged around the sensor A varies depending on the position at which the distortion is applied to the sensors B and C, depending on directions in which the distortion of, for example, the skin surface and the surface layer of the muscle is generated due to the body motion.

For this reason, in order to accurately detect the biological information at sensitivity of the portion due to the directionality of the strain, in the body motion noise reduction system of the first modification, the sensors B and C of a similar element are finely divided around the sensor A for detecting the biological information to sandwich the sensor A. Accordingly, the body motion noise reduction system of the modification can improve the detection sensitivity depending on the portion. Thus, the detection accuracy of the body motion noise reduction system can be enhanced.

A description is given below of some aspects according to the present disclosure.

### First Aspect

A biological information detector includes a biological information sensor to measure biological information, multiple body motion information sensors to measure body motion information, and a detection unit to detect the biological information based on an output of the biological information sensor and outputs of the multiple body motion information sensors.

### Second Aspect

In the biological information detector according to the first aspect, the detector includes an estimation unit and a detection unit. The estimation unit to estimate the body motion information at the position of the biological information sensor based on the outputs of the multiple body motion information sensors. The detection unit detects the biological information based on the output of the biological information sensor and the body motion information estimated by the estimation unit.

### Third Aspect

In the biological information detector according to the second aspect, two of the body motion information sensors among the multiple body motion information sensors are arranged to sandwich the biological information sensor.

### Fourth Aspect

In the biological information detector according to the third aspect, the estimation unit estimates the body motion information at the position of the biological information sensor based on the distance from the biological information sensor to each of the two body motion information sensors or the ratio of the distances between the two body motion information sensors and the biological information sensor.

### Fifth Aspect

In the biological information detector according to any one of the first to fourth aspect, the biological information sensor and the multiple body motion information sensors are divided from a same element or constituent elements separated from or integrated as a single component.

### Sixth Aspect

The biological information detector according to any one of the first to fifth aspect further includes a correction unit to detect a difference between an output of the biological information sensor and an output of the body motion information sensor at predetermined intervals and corrects the output of the biological information sensor.

### Seventh Aspect

A biological information processing device includes an estimation unit to estimate body motion information at a position of a biological information sensor to measure biological information, based on outputs of multiple body motion information sensors to measure the body motion information, and a detector to detect the biological information based on the output of the biological information sensor and the body motion information estimated by the estimation unit.

### Eighth Aspect

A biological information processing method includes processing a biological information, executed by a biological information processing device. The processing includes estimating body motion information and detecting the biological information. The estimating estimates body motion information at a position of a biological information sensor that measures biological information, based on outputs of multiple body motion information sensors that measure the body motion information. The detecting detects the biological information based on an output of the biological information sensor and the body motion information estimated by the estimation unit.

The above-described embodiments are illustrative and do not limit the present disclosure. Thus, numerous additional modifications and variations are possible in light of the above teachings. For example, elements and/or features of different illustrative embodiments may be combined with each other and/or substituted for each other within the scope of the present disclosure.

Each of the functions of the described embodiments may be implemented by one or more processing circuits or circuitry. Processing circuitry includes a programmed processor, as a processor includes circuitry. A processing circuit also includes devices such as an application specific integrated circuit (ASIC), a digital signal processor (DSP), a field programmable gate array (FPGA), and conventional circuit components arranged to perform the recited functions.

1. A biological information detector comprising:
multiple sensors to measure body motion of a body and output body motion information; and
a sensor between the multiple sensors to measure biological information of the body and output the biological information;
a detector to detect the biological information based on:
the biological information output from the sensor; and
the body motion information output from the multiple sensors.

2. The biological information detector according to claim 1,
wherein the detector includes circuitry configured to:
estimate the body motion information at a position of the sensor based on the body motion information output from the multiple sensors; and
detect the biological information based on the biological information output from the sensor and the body motion information at the position of the sensor.

3. The biological information detector according to claim 2,
wherein the multiple sensors include two sensors to measure body motion of a body,
the sensor is disposed between the two sensors,
the sensor is adjacent to each of the two sensors,
the sensor and the two sensors are arrayed in one direction, and
the sensor and the two sensors are arranged at predetermined interval with each other in the one direction.

4. The biological information detector according to claim 3,
wherein the circuitry is further configured to:
estimate the body motion information at the position of the sensor based on:
a distance from the sensor to each of the two sensors; or
a ratio of the distance from the sensor to one of the two sensors and the distance from the sensor to another of the two sensors.

5. The biological information detector according to claim 1,
wherein the sensor and the multiple sensors are:
division of the same element; or
multiple elements formed into a single component.

6. The biological information detector according to claim 1,
wherein the circuitry is further configured to detect a difference between the biological information output from the sensor and the body motion information output from each of the multiple sensors at a predetermined interval to correct the biological information output from the sensor.

7. A biological information processing device comprising circuitry configured to:
receive body motion information output from multiple sensors to measure body motion of a body at multiple positions;
receive biological information of the body output from the sensor to measure the biological information at a position between the multiple sensors;
estimate the body motion information at the position of the sensor based on the body motion information measured by the multiple sensors; and
detect the biological information based on the biological information measured by the sensor at the position and the body motion information estimated at the position.

8. A method for processing biological information, the method comprising:
measuring body motion of a body at multiple positions and outputting body motion information;
measuring biological information of the body at a position between the multiple positions and outputting the biological information;
estimating the body motion information at the position, at which the biological information is measured, based on the body motion information measured at the multiple positions; and
detecting the biological information based on the biological information measured at the position and the body motion information estimated at the position.

9. The biological information detector according to claim 1, further comprising an absorber or a convex-shaped medium between the multiple sensors.

10. The biological information detector according to claim 1, further comprising:
a groove between the multiple sensors; and
an absorber in the groove.
